(19) United States
(12) Patent Application Publication
(10) Pub. No.: US 2018/0182474 A1
(43) Pub. Date: Jun. 28, 2018

(54) SUSPECTED HIERARCHICAL CONDITION CATEGORY IDENTIFICATION

(71) Applicant: CERNER INNOVATION, INC., Kansas City, KS (US)

(72) Inventors: COLE ANTHONY ERDMANN, KANSAS CITY, MO (US); Jeff Christianson, KANSAS CITY, MO (US)

(21) Appl. No.: 15/856,932

(22) Filed: Dec. 28, 2017

Related U.S. Application Data
(60) Provisional application No. 62/439,769, filed on Dec. 28, 2016.

(51) Int. Cl. G16H 10/60; G06F 17/30

(52) U.S. Cl. CPC G16H 10/60; G06F 17/30303

(57) ABSTRACT

The present invention relates to methods and systems for utilizing hierarchical condition category suspected condition models to flag diagnoses for patients which have a high likelihood of requiring documentation in a current year based on previously recorded clinical information. A suspected HCC stratification for a patient may be generated and supporting and competing facts for the HCC stratification may be provided to drive further action for a patient.

### BACKGROUND

The present invention relates to developing processes for implementing systems and methods to improve data quality, particularly in the healthcare field. Hierarchical Condition Categories drive risk adjustment models in the healthcare setting, for example an institution may quantify its total risk by HCC, which drives payment to the institution, i.e. reimbursement to an institution is based on condition modeling. While the total population of an institution may be stratified in this way, reimbursement to an institution is only for patients under qualifying Centers for Medicare and Medicaid Services (CMS) plans. Standard HCC's known in the industry are defined by specific conditions in an attempt to group patients correctly with the correct associated medical billing codes so the correct reimbursement claims are filed; in this way HCC modeling is done by utilizing direct indicators. However, current methods can be inconsistent, cause gaps in information, or cause uncertainty as to the accuracy of billing codes or HCC groupings. For instance, one significant issue with conventional methods are inaccuracies in the underlying International Classification of Diseases (ICD) codes which directly map to HCC's. As such, current methods of utilizing HCC's to drive payment for a healthcare institution are not accurate enough to reliably ensure that members belonging to a set under an HCC should be there and are coded completely or accurately.

### SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

At a high level, embodiments of the present invention generally relate to developing and utilizing systems and processes to drive hierarchical condition category (HCC) identification for healthcare patient coding and hospital revenue cycle reimbursement. In particular, various embodiments relate to the implementation of HCC suspected condition models to drive improved patient coding and grouping and improved accuracy of filed reimbursement claims as a part of the healthcare revenue cycle. Embodiments of the present invention utilize a clinically driven approach to identify and correctly classify and/or code patients as part of the HCC methodology, and does this by employing HCC suspected condition models, e.g. a patient does not have a direct indicator, such as an ICD diagnosis code, that they should be grouped or coded one way, but certain clinical factors point to them being part of a condition group (i.e. an HCC group).

Additional objects, advantages, and novel features of the invention will be set forth in part in the description which follows, and in part will become apparent to those skilled in the art upon examination of the following, or may be learned by practice of the invention. This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter. The present invention is defined by the claims.

### BRIEF DESCRIPTION OF THE DRAWINGS

The features of the invention noted above are explained in more detail with reference to the embodiments illustrated in the attached drawing figures, in which like reference numerals denote like elements, in which FIGS. 1-4 illustrate embodiments of the present invention and in which:

FIG. 1 is a block diagram of an exemplary computing environment suitable to implement embodiments of the present invention;

FIG. 2 is a block diagram of an exemplary operating system utilizing HCC suspected condition identification in accordance with embodiments of the present invention;

FIG. 3 depicts an exemplary flow diagram illustrating methods of implementing HCC suspected condition identification, in accordance with embodiments of the present invention; and

FIG. 4 depicts an exemplary flow diagram illustrating methods of implementing HCC suspected condition identification, in accordance with embodiments of the present invention.

### DETAILED DESCRIPTION

The subject matter of the present invention is described with specificity herein to meet statutory requirements. However, the description itself is not intended to limit the scope of this patent. Rather, the inventors have contemplated that the claimed subject matter might also be embodied in other ways, to include different steps or combinations of steps similar to the ones described in this document, in conjunction with other present or future technologies. Moreover, although the terms “step” and/or “block” may be used herein to connote different elements of methods employed, the terms should not be interpreted as implying any particular order among or between various steps herein disclosed unless and except when the order of individual steps is explicitly described.

An important aspect of healthcare revenue cycle systems is accurately and efficiently coding patients to aid in the correct and complete filing of reimbursement claims to ensure proper payment back to a healthcare provider, for example in the context of Medicare and Medicaid services. Hierarchical Condition Categories (HCC) can be employed in healthcare revenue cycle systems to assist in correct coding, for example in condition or disease coding. As such the HCC coding model identifies individuals with illnesses qualifying for reimbursement and assigns risk factor scores to the person based on a combination of an individual patient's health conditions and demographic details. According to the HCC model, an individual patient's health conditions are identified by ICD-9 and ICD-10 diagnosis codes that are provided on claims, and in the risk adjustment model of services providing reimbursement, the diagnosis codes map to HCC's. Documentation of a patient's medical record by the provider must support the submitted diagnosis codes. HCC's utilizing ICD codes drive how risk adjustment is handled by healthcare systems, i.e. total risk can be quantified by HCC. HCC's are thus employed to adjust a given patient's risk versus others which in turn drives payment in the form of healthcare cost reimbursements. For example, fixed payments can be given per member based on an overall population.

One goal of healthcare modeling and managing data quality issues within clinical systems is to model or train a model to find members of a population who look like other members of a training set who are known to have a condition. Currently, however, ICD codes are not accurate enough to reliably identify members of such a training set who have a condition. As such, the systems and methods according to embodiments of the present invention enable more accurate coding and HCC identification for healthcare patient coding thus improving data quality within a clinical system and further allowing for the ability to better train predictive models.

Accordingly, aspects of the technology described herein are generally directed to methods and systems for improving coding and coding accuracy of patients to drive healthcare revenue cycles. For example, one aspect of healthcare modeling is to improve ICD coding accuracy through the use of clinically based models. Various embodiments of the present invention are directed to facilitating the design and utilization of hierarchical condition category models and algorithms to improve the accuracy and efficiency of patient diagnosis coding to drive risk adjustment, accurate reimbursement claim filing, and payment or collections. Such models can be, for example, based on unique combinations of clinical identifiers and benchmarking other condition identification algorithms. By utilizing HCC's created for specific conditions according to the present technology, a healthcare entity can (1) code appropriately on an annual basis (persistence coding) and (2) can identify gaps and indicate suspected coding (e.g. a patient is not flagged for persistence coding, but a patient can be identified as looking like they have a condition or disease even though they are not coded for that condition or disease). It should be noted that with respect to persistence coding, CMS requires annual coding for annual reimbursement. Accordingly, an HCC persistence algorithm is run and can indicate a patient has been previously coded for a particular condition or disease, but not this year, and therefore should be coded the same this year. An HCC suspected condition algorithm can also be used to see if a patient does not necessarily have a previous code for a given condition or disease, but certain clinical indications suggest the patient should be coded for the given condition or disease.

In various embodiments of the present invention, HCC persistence algorithms and HCC suspected condition algorithms can be run in conjunction with one another, supplementing each other and driving improved escalation of any actions needed to be taken by clinicians or coders, for example that a patient should be further evaluated based on some outcome of one of the algorithms, or that a patient should in fact be coded for a particular condition in the current year, or that a suspected condition indication should be reviewed.

HCC persistence and suspected algorithms can be configured to run based on various scenarios presented for any number of patients through data mined from one or more electronic health records (EHR) systems, patient access systems, revenue cycle systems, or any number of identified clinical systems. For example, various combinations of conditions may cause an HCC persistence algorithm to run and/or generate a flag or cause an HCC suspected algorithm to run and present an indication of a suspected condition to a clinician or coder.

In various embodiments, systems and methods described herein utilize conditional indications to run the persistence algorithm and/or run the suspected algorithm (and/or flag). Conditions used can include qualifying conditions (e.g. conditions coded or documented in the last two years, or one of the last two years, this time frame being configurable by institution) and satisfying conditions (e.g. conditions coded or documented in a current year to date). The persistence algorithm can flag previously coded or documented diagnosis or conditions that have not been coded (or documented) this year. The suspected algorithm can flag diagnosis or conditions that are suspected to be coded or documented this year based on any previously recorded diagnostics, labs, treatments, and the like. The output from the algorithms complement each other and present to a clinician or medical coding professional one or more recommendations or indications. Both the persistence and suspected algorithms depend on codes and data within the last two years as well as the current year to date to drive their output.

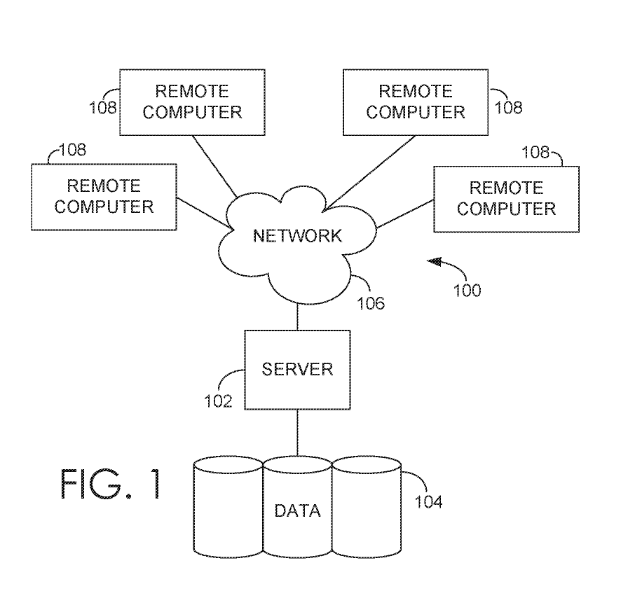

An exemplary computing environment suitable for use in implementing embodiments of the present invention is described below. FIG. 1 is an exemplary computing environment (e.g., medical-information computing-system environment) with which embodiments of the present invention may be implemented. The computing environment is illustrated and designated generally as reference numeral 100. The computing environment 100 is merely an example of one suitable computing environment and is not intended to suggest any limitation as to the scope of use or functionality of the invention. Neither should the computing environment 100 be interpreted as having any dependency or requirement relating to any single component or combination of components illustrated therein.

Embodiments of the present invention can be operational with numerous other purpose computing system environments or configurations. Examples of well-known computing systems, environments, and/or configurations that might be suitable for use with the present invention include personal computers, server computers, hand-held or laptop devices, multiprocessor systems, microprocessor-based systems, set top boxes, programmable consumer electronics, network PCs, minicomputers, mainframe computers, distributed computing environments that include any of the above-mentioned systems or devices, and the like.

Embodiments of the present invention can be described in the general context of computer-executable instructions, such as program modules, being executed by a computer. Exemplary program modules comprise routines, programs, objects, components, and data structures that perform particular tasks or implement particular abstract data types. The present invention can be practiced in distributed computing environments where tasks are performed by remote processing devices that are linked through a communications network. In a distributed computing environment, program modules can be located in association with local and/or remote computer storage media (e.g., memory storage devices).

With continued reference to FIG. 1, the computing environment 100 comprises a computing device in the form of a control server 102. Exemplary components of the control server 102 comprise a processing unit, internal system memory, and a suitable system bus for coupling various system components, including data store 104, with the control server 102. The system bus might be any of several types of bus structures, including a memory bus or memory controller, a peripheral bus, and a local bus, using any of a variety of bus architectures. Exemplary architectures comprise Industry Standard Architecture (ISA) bus, Micro Channel Architecture (MCA) bus, Enhanced ISA (EISA) bus, Video Electronic Standards Association (VESA) local bus, and Peripheral Component Interconnect (PCI) bus, also known as Mezzanine bus.

The control server 102 typically includes therein, or has access to, a variety of computer-readable media. Computer-readable media can be any available media that can be accessed by control server 102, and includes volatile and nonvolatile media, as well as removable and nonremovable media. By way of example, and not limitation, computer-readable media may comprise computer storage media and communication media. Computer storage media includes both volatile and nonvolatile, removable and non-removable media implemented in any method or technology for storage of information such as computer-readable instructions, data structures, program modules or other data. Computer storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can be accessed by control server 102. Communication media typically embodies computer-readable instructions, data structures, program modules or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared and other wireless media. Combinations of any of the above should also be included within the scope of computer-readable media.

The control server 102 can operate in a computer network 106 using logical connections to one or more remote computers 108. Remote computers 108 can be located at a variety of locations in a medical or research environment, including clinical laboratories (e.g., molecular diagnostic laboratories), hospitals and other inpatient settings, veterinary environments, ambulatory settings, medical billing and financial offices, hospital administration settings, home healthcare environments, and clinicians' offices. Clinicians or healthcare providers may comprise a treating physician or physicians; specialists such as surgeons, radiologists, cardiologists, and oncologists; emergency medical technicians; physicians' assistants; nurse practitioners; health coaches; nurses; nurses' aides; pharmacists; dieticians; microbiologists; laboratory experts; laboratory technologists; genetic counselors; researchers; veterinarians; students; and the like. The remote computers 108 can also be physically located in nontraditional medical care environments so that the entire healthcare community can be capable of integration on the network. The remote computers 108 can be personal computers, servers, routers, network PCs, peer devices, other common network nodes, or the like and can comprise some or all of the elements described above in relation to the control server 102. The devices can be personal digital assistants or other like devices.

Computer networks 106 comprise local area networks (LANs) and/or wide area networks (WANs). Such networking environments are commonplace in offices, enterprise-wide computer networks, intranets, and the Internet. When utilized in a WAN networking environment, the control server 102 can comprise a modem or other means for establishing communications over the WAN, such as the Internet. In a networking environment, program modules or portions thereof can be stored in association with the control server 102, the data store 104, or any of the remote computers 108. For example, various application programs may reside on the memory associated with any one or more of the remote computers 108. It will be appreciated by those of ordinary skill in the art that the network connections shown are exemplary and other means of establishing a communications link between the computers (e.g., control server 102 and remote computers 108) can be utilized.

In operation, an organization can enter commands and information into the control server 102 or convey the commands and information to the control server 102 via one or more of the remote computers 108 through input devices, such as a keyboard, a pointing device (commonly referred to as a mouse), a trackball, or a touch pad. Other input devices comprise microphones, satellite dishes, scanners, or the like. Commands and information can also be sent directly from a remote healthcare device to the control server 102. In addition to a monitor, the control server 102 and/or remote computers 108 can comprise other peripheral output devices, such as speakers and a printer.

Although many other internal components of the control server 102 and the remote computers 108 are not shown, such components and their interconnection are well known. Accordingly, additional details concerning the internal construction of the control server 102 and the remote computers 108 are not further disclosed herein.

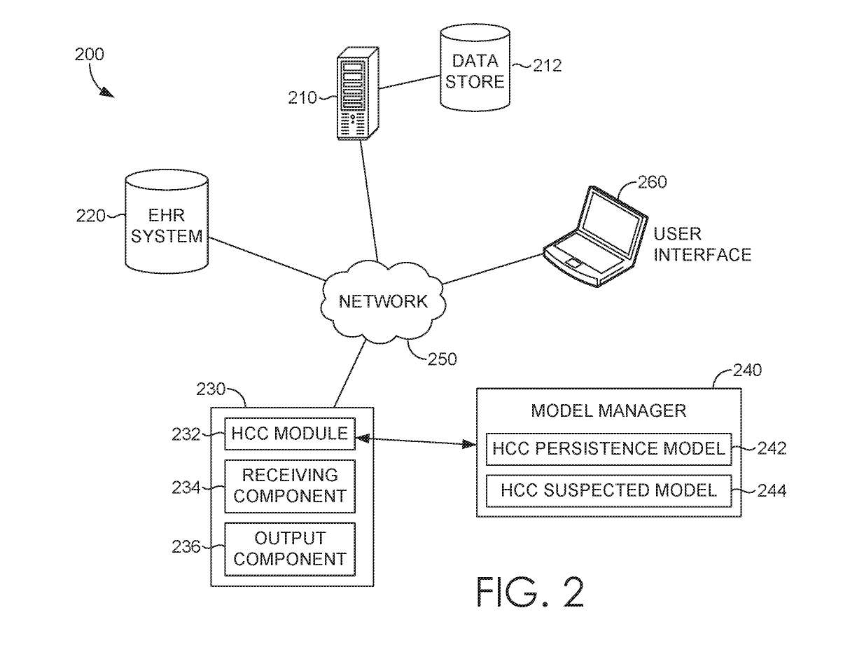

FIG. 2 provides a schematic diagram showing an exemplary HCC suspected condition identification system 200 in accordance with various embodiments of the present disclosure. The HCC suspected condition identification system 200 can include a computing system 210 and data store 212 in operable communication with one or more electronic health record (EHR) systems and a clinician or other interface 260 over a network 250.

The computing system 200 can include a software stack, optionally operating as a distributed system, configured to provide a number of services, components, or modules. The HCC suspected condition identification system 200 can include a receiving component 234, an HCC module 232, and an output component 236. The HCC module 232 may be in operable communication with a model manager 240 which can be configured to host and run one or more HCC persistence models 242 and one or more HCC suspected condition models 244.

The receiving component 234 is configured to receive healthcare data elements for one or more patients, the healthcare data elements comprising any number of healthcare data, including clinical data, prescription data, and/or billing data, or demographic data associated with a patient. In one aspect, the receiving component 234 can retrieve or mine data from one or more EHR systems 220 or one or more connected data stores 212. The receiving component 234 can be configured to mine and retrieve healthcare data for the system 200, for example over a predetermined measurement or analysis period. The measurement period can be broken into full or partial periods, for example, at least one full previous year or a year to date. The receiving component 234 can receive an input determining the measurement period, or the system 200 can automatically generate one or more measurement periods. As such, the receiving component 234 may only mine patient data based on that measurement period.

The model manager 240 can manage at least one HCC persistence model 242. In various embodiments, the HCC module 232 can implement at least one HCC persistence model 242. In some embodiments, the receiving component 234 receives a set of data elements for one or more patients, wherein the set of data elements include an ICD code or an HCC code within a given measurement period. Based on that set of data elements, the HCC persistence model 242 can determine if that code should persist into the current year for a given patient. For example, a patient has been previously been coded or billed for an HCC category and/or an associated ICD code and an indication is generated that the patient should again be coded or billed for an HCC category and/or associated ICD code.

The model manager 240 can manage at least one HCC suspected condition model 244, and the HCC module 232 can implement at least one HCC suspected condition model 244. A HCC suspected condition model 244 is configured to produce a suspected stratification for a patient for a condition category (e.g. Congestive Heart Failure). The HCC suspected condition model 244 is configured to run for a patient and determine one or more suspected stratifications, for example, highly suspected, moderately suspected, or not suspected. The HCC suspected condition model 244 can further be configured to indicate supporting facts for the determined stratification and/or competing facts that may indicate the patient may not belong in that group.

In accordance with implementations of the present invention, HCC suspected condition models 244 can be based on clinical data, for example unique groupings of clinical data and unique ranges for such data. When an HCC suspected condition model 244 runs, it compares a plurality of test criteria to the mined patient data elements, and if the set of data elements meets a previously determined or automatically determined threshold of test criteria, the HCC suspected condition model 244 will generate a suspected HCC stratification for the patient. The stratifications within an HCC suspected condition model 244 can be associated with a subgroup of test criteria (and thus define a set of supporting facts that may or may not be met). The stratifications within an HCC suspected condition model 244 can be separately associated with one or more potential competing facts or competing test criteria. Potential competing facts can be any data element associated with a patient (e.g. clinical information) that does not support persistence coding of a given diagnosis for that patient, or would tend to support a different diagnosis code.

In some instances the HCC suspected condition models 244 can include suppression criteria, and if there is a determination that any data element received for a patient meets the threshold suppression criteria, the system will suppress the HCC suspected condition model 244 from running and therefore suppress any output for that HCC suspected condition model 244.

The output component 236, can be configured to provide for display a suspected HCC stratification, any supporting criteria, or any competing criteria or facts. The output component may also display a recommendation for a clinician or coder to follow up for a patient, or verify/confirm the HCC or code, a previous HCC or code, or another conflicting HCC or code. The output component 236 can be configured to further automatically generate a recommendation for a patient and add it to his or her associated EHR or add a billing item to a worklist. For example, the output component 236 may flag a patient with suspicion such that a clinician must confirm or deny an associated HCC suspected stratification within the correct HCC suspected condition model 244.

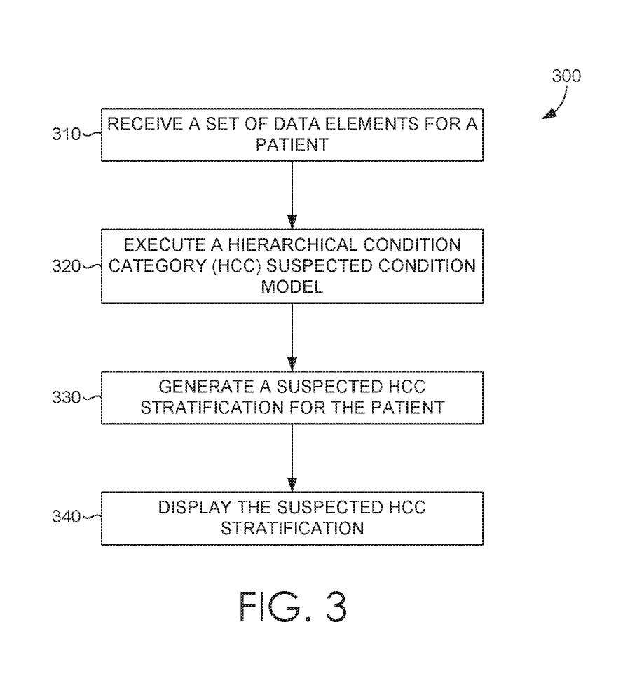

Turning now to FIG. 3, a flow diagram is depicted of an exemplary method 300 of implementing HCC suspected condition identification. At block 310 a set of data elements for a patient is received. The set of data elements can be within a predetermined measurement period, for example within the two full previous years. At block 320, at least one hierarchical condition category (HCC) suspected condition model is processed or otherwise executed by the system. The HCC condition model can comprise a plurality of test criteria. At block 330, a suspected HCC stratification for the patient is generated based on a determination that the received set of data elements meets a threshold of test criteria of the HCC suspected condition model. At block 340, the suspected HCC stratification is provided for display.

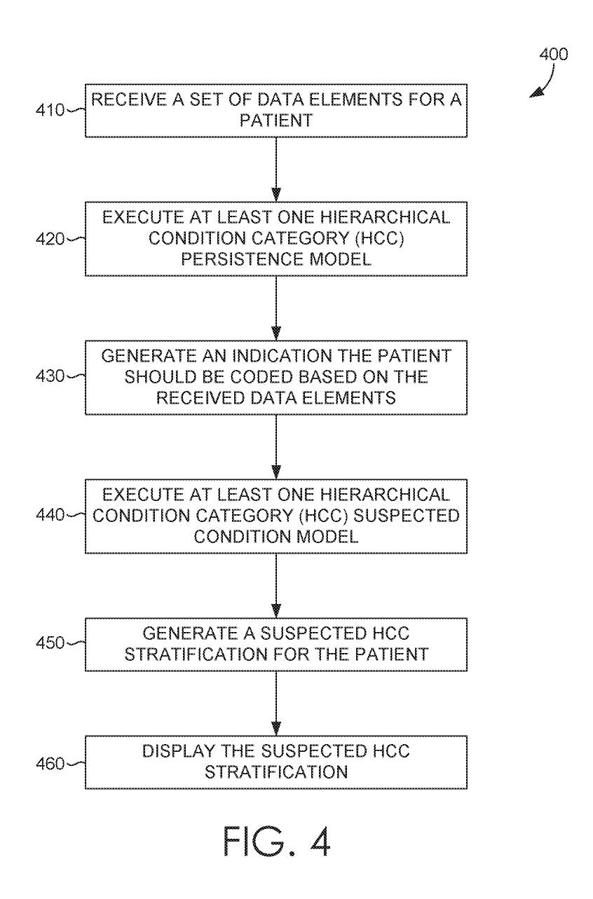

FIG. 4, depicts a flow diagram is of an exemplary method 400 of implementing HCC suspected condition identification. At block 410, a set of data elements is received for a patient, where the set of data elements is within a predetermined measurement period and the set of data elements comprises at least one previous HCC or ICD code. At 420, a HCC persistence model is processed or otherwise executed based on the previous HCC or ICD code and run against the patients received data. At block 430, the HCC persistence model generates an indication the patient should be coded based on the received previous HCC or ICD code. At block 440, at least one hierarchical condition category (HCC) suspected condition model is processed or otherwise executed by the system. The HCC suspected condition model can comprise a plurality of test criteria. At block 450, a suspected HCC stratification for the patient is generated based on a determination that the received set of data elements meets a threshold of test criteria of the HCC suspected condition model. At block 460, the suspected HCC stratification is provided for display.

### Examples

The following examples are merely for illustrative purposes and are not meant to limit the scope of the present invention. Table 1 below illustrates a number of exemplary cases in accordance with the present invention.

TABLE 1

| | | | | Run |
|---|---|---|---|---|
| | Qualifying Cond. | Satisfying Cond. | Persistence Flag. | Suspected. |
| 1 | Y | Y | N | N |
| 2 | Y | N | Y | Y |
| 3 | N | Y | N | N |
| 4 | N | N | N | Y |

Table 1 illustrates four exemplary scenarios for implementing the HCC persistence models and supplementing the outcomes generated with one of more HCC suspected condition models. The “Qualifying Condition” column denotes whether at least one or more conditions being analyzed for a given patient have been coded in the previous two years. The “Satisfying Condition” column denotes whether the same one or more conditions being analyzed for a given patient have been coded in the current year to date. Thus in Scenario 1, a module may run for a patient (e.g. HCC module 232 of FIG. 2) for a given condition and determine that both Qualifying and Satisfying conditions have been met. In this instance, the persistence model will not be triggered to produce a flag and the HCC suspected condition model will not be run.

In Scenario 2, it may be determined that one or more Qualifying Conditions are met, but in the current year, no corresponding Satisfying Condition is indicated. In this instance, a persistence model will be run and a persistence flag will be generated, i.e. the system indicates that there may be a gap or inaccuracy in one or more coded conditions for a given patient. In this case, the HCC suspected condition model will automatically run to supplement the generated persistence flag. If the HCC suspected condition model or algorithm generates a suspected flag, it may complement the persistence flag and inform on how a given patient is to be currently coded. In a first instance, if the clinical information for the patient (e.g. diagnostics, labs, treatments, etc.) is recent, the system can generate both supporting and competing facts to inform whether persistence coding is warranted. For example, in the case of diabetes (e.g. HCC 19 below), a patient has a persistence diagnosis of diabetes and the HCC suspected condition model indicates the patient's hemoglobin test (HbA1c) labs are still high and the patient is still taking insulin (i.e. supporting facts that support the persistence coding or persistent diagnosis); however the HCC suspected condition model also indicates the patient was pregnant at some specified point during the qualifying period (i.e. a potential competing fact against the persistent diagnosis). In another example, for chronic kidney disease (e.g. HCC 137 below), a patient has a persistent diagnosis that indicates chronic kidney disease, and the HCC suspected condition model indicates the patient's glomerular filtration rate is very low (i.e. a supporting fact for the persistent diagnosis), however the HCC suspected condition model also indicates the patient had a kidney transplant at a later event date (i.e. a potential competing fact against persistence coding that drive an indication for follow up, clinical or otherwise). In both examples, the HCC suspected condition model will output supporting facts that support persistence coding of the diagnosis (in other words the diagnosis should persist), but there are also potential competing facts that indicate that persistence coding may not be warranted and a different code may be required for the patient. Table 2 below illustrates this aspect.

TABLE 2

| | | | | | | |
|---|---|---|---|---|---|---|
| | Service | Suspected | | | Potential | |
| Persistent Diagnosis | Date | Stratification | Supporting Facts | Date | Competing Facts | Date |

HCC 19 Diabetes without Complications

| | | | | | | | |
|---|---|---|---|---|---|---|---|
| E08.9 | Diabetes mellitus | Nov. 15, 2015 | High | HbA1c = 9% | Nov. 15, 2015 | Pregnancy = TRUE | Mar. 5, 2015 |
| | due to underlying | | | HbA1c = 9.5% | Jan. 1, 2016 | | |
| | condition without | | | Insulin = TRUE | Jan. 15, 2016 | | |
| | complication | | | | | | |

HCC 137 Chronic Kidney Disease, Stage 4

| | | | | | | | |
|---|---|---|---|---|---|---|---|
| N18.4 | Chronic kidney | Mar. 1, 2015 | High | GFR = 12 | May 30, 15 | Kidney Transplant = | Oct. 15, 2015 |
| | disease, stage 4 | | | | | TRUE | |
| | (severe) | | | | | | |

In a second instance, a patient's clinical information (e.g. diagnostics, labs, treatments) and their timing may indicate a reason for ignoring persistence coding for the current year. For example, a persistence model shows a code or flag for drug induced diabetes from 2014, and the most recent values that are returned after the suspected model is run are high diabetes labs (RPG/FPG/HbA1c) during that same time period. Thus, it may be determined that the patient may no longer qualify for the particular diabetes HCC. Table 3 below illustrates this aspect.

TABLE 3
HCC 19 Diabetes without Complications

| | | Service | Suspected | | | Potential | |
|---|---|---|---|---|---|---|---|
| Persistent Diagnosis | | Date | Stratification | Supporting Facts | Date | Competing Facts | Date |
| E09.9 | Drug or chemical | Mar. 25, 2014 | High | RPG = 260 mg/dL | Mar. 1, 2014 | — | — |
| | induced diabetes | | | HbA1c = 8.5% | Mar. 1, 2014 | | |
| | mellitus without | | | | | | |
| | complications | | | | | | |

In a third instance, a patient's clinical information (e.g. diagnostics, labs, treatments) and their timing may indicate a reason for a different code under the same HCC. For example, the persistence model can generate a code or flag for drug induced diabetes from 2014, but when the suspected model is run, processed, or otherwise executed, values are generated showing high diabetes labs (RPG/FPG) for 2016. Thus, it may be determined that a patient has developed chronic diabetes since the drug induced episode, therefore qualifying for the diabetes HCC, but not the same code that was flagged by the persistence model. Table 4 below illustrates this aspect.

TABLE 4
HCC 19 Diabetes without Complications

| | | Service | Suspected | | | Potential | |
|---|---|---|---|---|---|---|---|
| Persistent Diagnosis | | Date | Stratification | Supporting Facts | Date | Competing Facts | Date |
| E09.9 | Drug or chemical | Mar. 25, 2014 | High | RPG = 260 mg/dL | Sep. 1, 2015 | — | — |
| | induced diabetes | | | HbA1c = 8.5% | Jan. 15, 2016 | | |
| | mellitus without | | | | | | |
| | complications | | | | | | |

Referring back to Table 1, in scenario 3, no qualifying conditions are indicated, but satisfying conditions are met and/or indicated. In this scenario, this indication signifies that one or more conditions have been coded for this year.

Lastly, in scenario 4, no qualifying or satisfying conditions are indicated, and therefore no flag is generated by the persistence model. However, if appropriate clinical information (e.g. diagnostics, labs, treatments) for a patient is found, the HCC suspected condition model will give a flag for an appropriate HCC. In this scenario, the system may recognize that it looks like an HCC should be documented but has not been since prior to the qualifying period (or ever)—either a documentation gap longer than the period or a recently developed condition. If no suspected flag is generated, then an HCC will not be returned. Table 5 below illustrates this aspect.

TABLE 5

| | | | | | | |
|---|---|---|---|---|---|---|
| | Service | Suspected | | | Potential | |
| Persistent Diagnosis | Date | Stratification | Supporting Facts | Date | Competing Facts | Date |

HCC 137 Chronic Kidney Disease, Stage 4

| | | | | | | | |
|---|---|---|---|---|---|---|---|
| N18.4 | Chronic kidney | Mar. 1, 2015 | High | GFR = 12 | May 30, 2015 | — | — |
| | disease, stage 4 | | | | | | |
| | (severe) | | | | | | |

HCC 19 Diabetes without Complications

| | | | | | | | |
|---|---|---|---|---|---|---|---|
| — | — | — | High | HbA1c = 9% | Nov. 15, 2015 | — | — |
| | | | | FPG = 160 mg/dL | Nov. 15, 2015 | | |

It will also be appreciated that in the instance no flag is generated by the persistence model or the suspected model, and there is no high-percentage clinical information on record to confirm a diagnosis, a code recognized as a qualifying condition may still be valid to persist into the current year. In this instance a verification may be run and/or presented to a clinician to verify or confirm the diagnosis code.

Many variations can be made to the illustrated embodiment of the present invention without departing from the scope of the present invention. Such modifications are within the scope of the present invention. Other modifications would be readily apparent to one of ordinary skill in the art, but would not depart from the scope of the present invention.

From the foregoing it will be seen that this invention is one well adapted to attain all ends and objects hereinabove set forth together with the other advantages which are obvious and which are inherent to the structure. It will be understood that certain features and subcombinations are of utility and may be employed without reference to other features and subcombinations. This is contemplated by and is within the scope of the invention.

Since many possible embodiments may be made of the invention without departing from the scope thereof, it is to be understood that all matter herein set forth or shown in the accompanying drawings is to be interpreted as illustrative of applications of the principles of this invention, and not in a limiting sense.

What is claimed is:

1. A system for improving data quality in a clinical system, the system comprising:
a computer store containing clinical data elements for a patient; and
a computer server at the clinical system, the computer server logically coupled to the computer store and configured to:
receive a set of clinical data elements for at least one patient, wherein the set of clinical data elements are measured within a predetermined measurement period;
execute at least one hierarchical condition category (HCC) suspected condition model, the HCC suspected condition model comprising a plurality of test criteria;
generate a suspected HCC stratification for the at least one patient based on a determination that the received set of clinical data elements meets a threshold of test criteria of the HCC suspected condition model; and
provide for display the suspected HCC stratification.

2. The system of claim 1, wherein the suspected HCC stratification is at least one of: highly suspected, moderately suspected, or not suspected.

3. The system of claim 2, wherein each suspected HCC stratification is associated with a subgroup of the plurality of test criteria, the subgroup of test criteria defining a set of supporting facts.

4. The system of claim 2, wherein each suspected HCC stratification is associated with zero or more competing facts.

5. The system of claim 3, further comprising providing for display at least one supporting fact simultaneously with the suspected HCC stratification.

6. The system of claim 4, further comprising providing for display at least zero or more competing facts simultaneously with the suspected HCC stratification based on a determination that the set of clinical data elements meets a threshold for the zero or more competing facts.

7. The system of claim 1, wherein the HCC suspected condition model is associated with zero or more suppression criteria.

8. The system of claim 7, further comprising suppressing the suspected HCC stratification based on a determination that the set of clinical data elements meets a threshold of suppression criteria.

9. The system of claim 1, further configured to automatically provide a recommendation based on the generated HCC stratification.

10. The system of claim 1, further configured to execute, by a suspected condition identification system, at least a second hierarchical condition category (HCC) suspected condition model, the second HCC suspected condition model comprising a plurality of test criteria;
generate at least a second suspected HCC stratification for the at least one patient based on a determination that the set of clinical data elements meets a threshold of test criteria of the second HCC suspected condition model; and
provide for display the second suspected HCC stratification.

11. The system of claim 4, wherein the test criteria or the competing facts or both are based on clinical indicators.

12. A computer-implemented method for generating a suspected HCC stratification, comprising:
receiving a set of data elements for at least one patient, wherein the set of data elements are within a predetermined measurement period and the set of data elements comprise at least one ICD code;
executing at least one hierarchical condition category (HCC) persistence model;
generating by the HCC persistence model an indication the at least one patient should be coded based on the received ICD code;
executing at least one hierarchical condition category (HCC) suspected condition model, the HCC suspected condition model comprising a plurality of test criteria;
generating a suspected HCC stratification for the at least one patient based on a determination that the received set of data elements meets a threshold of test criteria of the HCC suspected condition model; and
providing for display the suspected HCC stratification.

13. The method of claim 12, wherein each suspected HCC stratification is associated with a subgroup of the plurality of test criteria, the subgroup of test criteria defining a set of supporting facts.

14. The method of claim 12, wherein each suspected HCC stratification is associated with zero or more competing facts.

15. The method of claim 13, further comprising determining the indication the at least one patient should be coded based on the received ICD code is valid.

16. The method of claim 13, further comprising determining the indication the at least one patient should be coded based on the received ICD code is invalid.

17. The method of claim 13, further comprising determining the at least one patient should be coded based on an ICD code that is different than the received ICD code.

18. The method of claim 14, wherein the test criteria or the competing facts or both are based on clinical indicators.

19. One or more computer storage media, having instructions stored thereon that, when executed by one or more processors of a computing system, cause the computing system to:
receive a set of data elements for at least one patient, wherein the set of data elements are measured within a predetermined measurement period;
execute at least one hierarchical condition category (HCC) suspected condition model, the HCC suspected condition model comprising a plurality of test criteria;
generate an indication that no suspected HCC stratification exists for the at least one patient based on a determination that the received set of data elements does not meet a threshold of test criteria of the HCC suspected condition model; and
provide for display the suspected HCC stratification indication.

20. The computer storage media of claim 19, wherein the instructions further cause a notification to be generated to confirm if a valid code exists to indicate persistence coding.
